(19) United States
(12) Patent Application Publication
(10) Pub. No.: US 2009/0017096 A1
(43) Pub. Date: Jan. 15, 2009

(54) POROUS NON-BIODEGRADABLE HYDROGEL ADMIXED WITH A CHEMOATTRACTANT FOR TISSUE REPLACEMENT

(76) Inventors: Anthony Lowman, Wallingford, PA (US); Suzanne A. Maher, New York, NY (US); Lorenzo Pio Serino, Reggello (IT); Kara Spiller, Philadelphia, PA (US); Peter A. Torzilli, Ridgefield, CT (US)

Correspondence Address: MORGAN & FINNEGAN, L.L.P., 3 WORLD FINANCIAL CENTER, NEW YORK, NY, 10281-2101, US

(21) Appl. No.: 11/997,453

(22) PCT Filed: Aug. 15, 2006
(86) PCT No.: PCT/US06/31843

§ 371 Date: Sep. 3, 2008

Related U.S. Application Data
(60) Provisional application No. 60/708,442, filed on Aug. 15, 2005.

(51) Int. Cl. A61L 27/58

(52) U.S. Cl. 424/426

(57) ABSTRACT

A non-biodegradable hydrogel matrix containing microspheres of a biodegradable polymer for the purpose of treating, repairing, or replacing damaged biological tissue is described. The biodegradable phase can be admixed with a chemoattractant. Examples of degradable polymers include degradable polyesters such as 50:50 PLA:PGA, the degradation profiles of which are well characterized. The matrix is permanently inserted into a tissue defect to provide mechanical support before, during, and after tissue ingrowth.

This application claims priority to provisional patent application U.S. 60/708,442, filed Aug. 15, 2005, which is incorporated by reference.

### BACKGROUND OF THE INVENTION

1. Field of the Invention

The invention relates to compositions for the treatment, repair, and replacement of in vivo tissue. In particular, the invention relates to permanent hydrogel implants that facilitate in vivo tissue ingrowth and integration.

2. Description of Relevant Art

Approximately 20 million individuals in the United States have been diagnosed with arthritis (Benson et al, “Current estimates from the National Health Interview Survey, 1995” Vital Health Stat, vol. 10, pages 1-428, 1998; Jackson et al, “Symptomatic articular cartilage degeneration: the impact in the new millennium” Clin. Orthop., vol. 391 Supp, pages S14-25; October 2001). This accounts for as many as 39 million physician visits and 500,000 hospitalizations per year at a cost of approximately $65 billion including direct medical costs of $15 billion/year (Centers for Disease Control and Prevention, “Targeting Arthritis: The Nation's Leading Cause of Disability” At-A-Glance, vol. 2, 1999, available at <http://www.cdc.gov/nccdphp/art-aag.htm>; Praemer et al, Muscoskeletal Conditions in the United States, American Academy of Orthopaedic Surgeons (publisher), pages 1-79, 1999). Articular cartilage damage is thus one of the most expensive of the debilitating non-life threatening diseases in the United States. The ability to repair cartilage defects with a synthetic material that mimics the function of articular cartilage and thus prevent or limit the further progression of the arthritis would impact the management of this disease tremendously.

Permanent implants, made from metals and plastics for total joint replacement, suffer from compatibility problems. Poor integration at the implant site can lead to bone loss over time and possible mechanical failure. Various attempts have been made to provide biodegradable implants which will be completely replaced by organic tissue over time. For example, U.S. Pat. No. 5,607,474 describes a multi-phase bioerodible implant to provide interim support to a diseased or damaged area while the tissue is being regenerated. Similarly, U.S. Pat. No. 6,852,330 describes bioresorbable scaffolds for implantation, where the ability of the scaffold material to resorb in a timely fashion is critical. The basic principle of this type of tissue engineering is to utilize a bioactive, biocompatible, and biodegradable scaffold to promote cellular differentiation and matrix generation within a defect leading to a structurally and mechanically sound repair tissue. However, thus far, no tissue engineered construct has been produced which successfully recreates the mechanical response of the intact tissue that it is intended to replace. Furthermore, the challenge of integrating the host tissue and the engineered construct has not been resolved.

Therefore, one object of the present invention is to provide tissue implants which recreate the functional response of intact tissue before, during, and after tissue ingrowth. A further object of the present invention is to provide tissue implants with compatible microspheres, which degrade to form porous structures leading to tissue ingrowth and successful and permanent integration of the implant into the tissue. These microspheres may be seeded with chemoattractants and/or growth factors to encourage cellular migration, cell proliferation and matrix generation.

### SUMMARY OF THE INVENTION

Compositions according to the invention are a non-biodegradable matrix which contains biodegradable components of the implant to facilitate in vivo tissue ingrowth and integration. Compositions according to the invention are permanently placed in a defect, damaged site or worn away tissue to replace or augment the load-bearing tissues such as cartilage, bone, ligaments, tendons, and menisci, minimally load bearing tissues such as the bladder and blood vessels, and non-loading tissues such as lung and liver.

### BRIEF DESCRIPTION OF THE DRAWINGS

FIG. 1 is a diagram showing insertion of an implant into a cartilage and bone defect, with subsequent ingrowth.

FIG. 2 is a diagram showing layers of a more porous hydrogel material placed on the surface of a stiffer base hydrogel material providing structural integrity.

FIG. 3 is an SEM at 40× magnification of a hydrogel layered construct showing a well-integrated interface between a porous surface layer and a non-porous base layer.

FIG. 4 is a diagram showing a multilayered hydrogel—where porosity, permeability and modulus vary through the depth to more closely mimic the mechanical behavior of the adjacent tissue.

FIG. 5 is a photograph of microspheres made according to Example 1.

FIG. 6 is an SEM at 250× magnification of a porous microsphere-seeded implant according to Example 2.

FIG. 7 shows the compressive modulus of the hydrogels of Example 2.

FIG. 8 shows a cross section of the transverse plane of a sample made with 10 vol % ethyl acetate and stirred at 300 rpm from Example 2.

FIG. 9 shows a sample made with 2 wt % PLGA and 25 vol % dichloromethane according to Example 3.

FIG. 10 shows the compressive modulus of hydrogels from Example 3.

FIG. 11 shows a sample made with 5 wt % microparticles according to Example 4.

FIG. 12 shows a sample made with 2 wt % microparticles and 25 vol % dichloromethane according to Example 5.

FIG. 13 shows SEM images of 10%, 20%, 50% & 75% PLGA hydrogels according to the supplemental information for Example 2.

FIG. 14 shows Dynamic Modulus vs. strain as a function of percent PLGA according to the supplemental information for Example 2.

FIG. 15 shows 2-Week, Chondrocyte-seeded hydrogel (static culture), vertical-sections; Top row is 50% PLGA; Bottom row images are of 75% PLGA samples, according to the supplemental information for Example 2.

### DETAILED DESCRIPTION OF THE INVENTION

Compositions according to the present invention are biocompatible compositions for treating, repairing, or replacing biological tissue comprising a non-biodegradable porous hydrogel and a biodegradable polymer. The biodegradable polymer is preferably in the form of microparticles (e.g. microspheres) which contain a chemokine and/or growth factor. The degradable phase will facilitate cellular ingress, matrix generation, and interfacial integration; while the non-degradable matrix will provide immediate and sustainable functional (e.g. load bearing) characteristics. After cellular ingress and matrix deposition, a composite synthetic/biological structure will exist. The invention is particularly suited to repair focal cartilage defects, but is not limited to focal cartilage defects since it may also be useful for repairing cartilage in non-articular regions. Methods of making compositions according to the invention and of treating, repairing, and replacing biological tissue are also encompassed.

Examples of hydrogel material include polyvinylalcohol (PVA), which may be physically cross-linked by partial crystallization of the chain. Such hydrogels are described, for example, in U.S. Pat. No. 4,663,358. PVA hydrogels are also described in U.S. Pat. No. 5,981,826. Other examples are hydrogels based on segmented polyurethanes or polyureas, an example of which is described in U.S. Pat. No. 5,688,855. The patents mentioned above are herein incorporated by reference in their entirety. The hydrogels according to the invention are non-biodegradable and porous. Preferably, the hydrogel has open-cell pores, allowing for ingrowth of the surrounding biological tissue. Other useful polymers include polyacrylate such as poly(acrylic acid), poly(methacrylic acid) and poly(hydroxyethyl methacrylate), polyacrylamides, polyethylene oxide and polyvinyl pyrrolidones (PVP). PVA can be blended with PVP with amounts of about 0.5 to about 5% to induce stability in the PVA network. The addition of PVP has demonstrated reduced in vitro dissolution (see Thomas, J. et al., “Novel associated hydrogel for nucleus pulposus replacement,” J. Biomed. Mater. Res. A., vol. 67A, issue 4, pages 1329-37, 2003).

Poly(vinyl alcohol) useful for the invention is typically obtained as a dry powder or crystal, with properties that can vary based molecular weight. Thus, the molecular weight of the poly(vinyl alcohol) can be chosen depending upon the particular application envisioned for the hydrogel. Generally, increasing the molecular weight of the poly(vinyl alcohol) increases the mechanical properties, such as the tensile, compressive, shear and bulk ultimate strengths, stiffness and modulus, and thereby improves the functional properties of the hydrogel as a support matrix. Poly(vinyl alcohol) having an average molecular weight of from about 25,000 to about 186,000 may be preferred for practicing the invention, depending on the properties of the tissue at the treatment site.

The mechanical properties of the hydrogel can be measured using several test methods. Load-deformation (stress-strain) tests (tensile, compression, shear) can be performed to measure the Young's (E) and instantaneous (E at 2 sec) mechanical stiffness and modulus (linear, elastic, tangent) [Charlebois M, McKee M D, Buschmann M D. “Nonlinear tensile properties of bovine articular cartilage and their variation with age and depth.” Journal of Biomechanical Engineering 2004; 126:129-137; Freeman M, Kempson G, Swanson S. “Variation in the physico-chemical and mechanical properties of human articular cartilage. II. Mechanical properties.” In: Kenedi R, editor. Perspectives in Biomedical Engineering: MacMillan, Strathclyde, Glasgow, 1972; 157-160; Kempson G, Freeman M, Swanson S. “The determination of a creep modulus for articular cartilage from indentation tests on the human femoral head.” Journal of Biomechanics 1971; 4:239-250. Kempson G, Muir H, Pollard C, Tuke M. “The tensile properties of the cartilage of human femoral condyles related to the content of collagen and glycosaminoclycans.” Biochimica et Biophysica Acta 1973; 297:456-472 Mizrahi J, Maroudas A, Lanir Y, Ziv I, Webber T J. “The “instantaneous” deformation of cartilage: effects of collagen fiber orientation and osmotic stress.” Biorheology 1986; 23:311-330]. Creep and relaxation tests can be performed to measure the time-dependent or viscoelastic mechanical properties of the hydrogel, such as the permeability (k), equilibrium aggregate modulus (Ha) and Poisson's ratio (v) [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Kempson G, Freeman M, Swanson S. “The determination of a creep modulus for articular cartilage from indentation tests on the human femoral head.” Journal of Biomechanics 1971; 4:239-250, Goldsmith A A J, Clift S E. “Investigation into the biphasic properties of a hydrogel for use in a cushion form replacement joint.” Journal of Biomechanical Engineering 1998; 120:362-369; Gu W Y, Yao H, Huang C Y, Cheung H S. “New insight into deformation-dependent hydraulic permeability of gels and cartilage, and dynamic behavior of agarose gels in confined compression.” Journal of Biomechanics 2003; 36:593-598; Mow V, Kuei S, Lai W, Armstrong C. “Biphasic creep and stress relaxation of articular cartilage in compression: Theory and experiments.” Journal of Biomechanical Engineering 1980; 102:73-84; Nettles D L, Vail T P, Morgan M T, Grinstaff M W, Setton L A. “Photocrosslinkable hyaluronan as a scaffold for articular cartilage repair.” Annals of Biomedical Engineering 2004; 32:391-397; Torzilli P, Depres A, McKibben D, Chan B, Co F, Wenz J, Carr J. “A device for measuring the compressive properties of thin specimens.” ASME Advances in Bioengineering 1988; BED-8:179-180]. The dynamic mechanical properties (modulus, E_{dyn}) can be measured by performing load-deformation tests at multiple loading and deformation frequencies, typically 0.001 to 100 Hz or cycles per second [Kisiday J D, Jin M, DiMicco M A, Kurz B, Grodzinsky A J. “Effects of dynamic compressive loading on chondrocyte biosynthesis in self-assembling peptide scaffolds.” J Biomechanics 2004; 37:595-604; Milentijevic D, Helfet D L, Torzilli P A. “Influence of stress magnitude on water loss and chondrocyte viability in impacted articular cartilage.” Journal of Biomechanical Engineering 2003; 125(5):594-601 Milentijevic D, Torzilli P A. “Influence of stress rate on water loss, matrix deformation and chondrocyte viability in impacted articular cartilage.” Journal of Biomechanics 2005; 38(3):493-502; Torzilli P. “Mechanical response of articular cartilage to an oscillating load.” Mechanics Research Communications 1984; 11:75-82]. The bulk modulus (K) can be measured by the stress-volume change of the hydrogel when subjected to an osmotic compression [Chen S S, Falcovitz Y H, Schneiderman R, Maroudas A, Sah R L. “Depth-dependent compressive properties of normal aged human femoral head articular cartilage: relationship to fixed charge density.” Osteoarthritis and Cartilage 2001; 9(5):561-5]. These mechanical properties will depend on the biomaterial or biochemical properties of the hydrogel, such as the water content and fixed-charged density (FCD). The water content of the hydrogel can be measured by measuring the wet and dry weights of the hydrogel [Torzilli P, Askari E, Jenkins J. “Water content and solute diffusion properties of articular cartilage.” In: Mow V R, A, Woo, S L-Y, editor. Biomehanics of Diarthroidial Joints. New York: Springer-Verlag; 1990. p 363-390], and the fixed-charge density determined by measuring anion (Cl⁻) or cation (Na⁺) equilibrium tracer concentration [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Freeman M, Kempson G, Swanson S. “Variation in the physico-chemical and mechanical properties of human articular cartilage. II. Mechanical properties.” In: Kenedi R, editor. Perspectives in Biomedical Engineering: MacMillan, Strathclyde, Glasgow, 1972; 157-160; Maroudas A, Thomas H “A simple physicochemical micromethod for determining fixed anionic groups in connective tissue.” Biochimica et Biophysica Acta 1970; 215:214-216]. In applications to replace tissue such as cartilage, lower molecular weight poly(vinyl alcohol) can be employed because lower tensile strength and lower tensile stiffness may be desirable.

The diluent mixed with the polyvinyl alcohol is preferably deionized and ultra filtered water to minimize the potential for any contamination of the polyvinyl alcohol. The mixture is preferably prepared by mixing from about 1 to about 50 parts by weight polyvinyl alcohol with about 99 to about 50 parts by weight water. A mixture can be obtained by mixing from about 10 to about 20 parts polyvinyl alcohol with from about 80 to about 90 parts by weight water, and an especially preferred mixture is obtained by mixing about 15 parts polyvinyl alcohol with about 85 parts by weight water. Isotonic saline (0.9% by weight NaCl, 99.1% water) or an isotonic buffered saline may be substituted for water to prevent osmotic imbalances between the material and surrounding tissues if necessary.

The concentration of the polyvinyl alcohol contributes to the mechanical properties of the hydrogel, and can thus be chosen depending upon the mechanical properties of the material one desires to obtain. A hydrogel implant to recreate the functional properties of articular cartilage will preferably have a range of mechanical properties similar to those found in human articular cartilage. Thus the mechanical properties of the hydrogel implant would be within the range of one or more of the following mechanical properties of articular cartilage: Young's (elastic) and instantaneous compressive modulus (E at 2 sec)=0.1×10⁶ to 1.0×10⁶ Newtons per square meter (N/m² or Pascals, Pa; 10⁶ Pa=MPa) [Armstrong C, Mow V. “Variations in the intrinsic mechanical properties of human articular cartilage with age, degeneration and water content.” Journal of Bone and Joint Surgery 1982; 64A:88-94; Athanaasiou K, Rosenwasser M, Buckwalter J, Malinin T, Mow V. “Interspecies comparisons of in situ intrinsic mechanical properties of distal femoral cartilage.” Journal of Orthopaedic Research 1991; 9:330-340; Charlebois M, McKee M D, Buschmann M D. “Nonlinear tensile properties of bovine articular cartilage and their variation with age and depth.” Journal of Biomechanical Engineering 2004; 126:129-137; Freeman M, Kempson G, Swanson S. “Variation in the physico-chemical and mechanical properties of human articular cartilage. II. Mechanical properties.” In: Kenedi R, editor. Perspectives in Biomedical Engineering: MacMillan, Strathclyde, Glasgow, 1972; 157-160; Kempson G, Freeman M, Swanson S. “The determination of a creep modulus for articular cartilage from indentation tests on the human femoral head.” Journal of Biomechanics 1971; 4:239-250; Kempson G, Muir H, Pollard C, Tuke M. “The tensile properties of the cartilage of human femoral condyles related to the content of collagen and glycosaminoclycans.” Biochimica et Biophysica Acta 1973; 297:456-472; Milentijevic D, Helfet D L, Torzilli P A. “Influence of stress magnitude on water loss and chondrocyte viability in impacted articular cartilage.” Journal of Biomechanical Engineering 2003; 125(5):594-601; Milentijevic D, Torzilli P A. “Influence of stress rate on water loss, matrix deformation and chondrocyte viability in impacted articular cartilage.” Journal of Biomechanics 2005; 38(3):493-502]; permeability (k)=0.1 to 5.0×10⁻¹⁵ m⁴/Ns [Armstrong C, Mow V. “Variations in the intrinsic mechanical properties of human articular cartilage with age, degeneration and water content.” Journal of Bone and Joint Surgery 1982; 64A:88-94; Athanaasiou K, Rosenwasser M, Buckwalter J, Malinin T, Mow V. “Interspecies comparisons of in situ intrinsic mechanical properties of distal femoral cartilage.” Journal of Orthopaedic Research 1991; 9:330-340; Gu W Y, Yao H, Huang C Y, Cheung H S. “New insight into deformation-dependent hydraulic permeability of gels and cartilage, and dynamic behavior of agarose gels in confined compression.” Journal of Biomechanics 2003; 36:593-598; Mow V, Kuei S, Lai W, Armstrong C. “Biphasic creep and stress relaxation of articular cartilage in compression: Theory and experiments.” Journal of Biomechanical Engineering 1980; 102:73-84]; equilibrium aggregate modulus (Ha)=0.01 to 10.0 MPa; Poisson's ratio (v)=0.0 to 0.5 [Armstrong C, Mow V. “Variations in the intrinsic mechanical properties of human articular cartilage with age, degeneration and water content.” Journal of Bone and Joint Surgery 1982; 64A:88-94; Athanaasiou K, Rosenwasser M, Buckwalter J, Malinin T, Mow V. “Interspecies comparisons of in situ intrinsic mechanical properties of distal femoral cartilage.” Journal of Orthopaedic Research 1991; 9:330-340; Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Mow V, Kuei S, Lai W, Armstrong C. “Biphasic creep and stress relaxation of articular cartilage in compression: Theory and experiments.” Journal of Biomechanical Engineering 1980; 102:73-84; O'Connor P, Orford C R, Gardner D L. “Differential response to compressive loads of zones of canine hyaline articular cartilage: micromechanical, light and electron microscopic studies.” Annuals of Rheumatic Diseases 1988; 47(5):414-420; Torzilli P. “The lubrication of human joints: a review.” In: Fleming D F, B N, editor. Handbook of Engineering in Medicine and Biology. Ohio: CRC Press; 1976. p 225-251; Torzilli P. “Measurement of the compressive properties of thin cartilage slices: evaluating tissue inhomogeneity.” In: Maroudas A K, K, editor. Cartilage Methods. New York: Academic Press; 1990. p 304-308; Wang C C, Hung C T, Mow V C. “An analysis of the effects of depth-dependent aggregate modulus on articular cartilage stress-relaxation behavior in compression.” Journal of Biomechanics 2001; 34(1):75-84]; dynamic mechanical properties (modulus, E_{dyn})=0.1 to 1000 MPa [Gu W Y, Yao H, Huang C Y, Cheung H S. “New insight into deformation-dependent hydraulic permeability of gels and cartilage, and dynamic behavior of agarose gels in confined compression.” Journal of Biomechanics 2003, 36:593-598; Milentijevic D, Helfet D L, Torzilli P A. “Influence of stress magnitude on water loss and chondrocyte viability in impacted articular cartilage.” Journal of Biomechanical Engineering 2003; 125(5):594-601, Milentijevic D, Torzilli P A. “Influence of stress rate on water loss, matrix deformation and chondrocyte viability in impacted articular cartilage.” Journal of Biomechanics 2005; 38(3):493-502; Torzilli P. “Mechanical response of articular cartilage to an oscillating load.” Mechanics Research Communications 1984; 11:75-82]; bulk modulus (K)=0.1 to 10.0 MPa. The hydrogel biomaterial properties necessary to recreate the functional properties of human articular cartilage are preferably similar to those for articular cartilage; water content=70 to 85% [Brocklehurst R, Bayliss M, Maroudas A, Coysh H, Freeman M, Revell P A. “The composition of normal and osteoarthritic articular cartilage from human knee joints.” Journal Bone and Joint Surgery 1984; 66A:95-106; Maroudas A. “Physical chemistry of articular cartilage and the intervertebral disc.” In; Sokoloff, L., editor, The Joints and Synovial Fluid, Academic Press, New York, 1980, pp. 240-293; Maroudas A. “Variations in the physico-chemical and mechanical properties of human articular cartilage. 1. Physico-chemical properties.” In: Kenedi, R. editor, Perspective in Biomedical Engineering, MacMillan, Strathclyde, 1972, pp. 153-156; Maroudas A, Bayliss M T, Venn M F. “Further studies on the composition of human femoral head cartilage.” Annals of Rheumatic Diseases 1980; 39:514-523; Torzilli P, Askari E, Jenkins J. “Water content and solute diffusion properties of articular cartilage.” In: Mow V R, A; Woo, S L-Y, editor. Biomechanics of Diarthroidial Joints. New York: Springer-Verlag; 1990. p 363-390; Venn M, Maroudas A. “Chemical composition and swelling of normal and osteoarthrotic femoral head cartilage. I. Chemical composition.” Annals of Rheumatic Diseases 1977; 36:121-129]; fixed-charge density (FCD)=0.1 to 0.2 mEq/gm tissue H₂0 [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Freeman M, Kempson G, Swanson S. “Variation in the physico-chemical and mechanical properties of human articular cartilage. II. Mechanical properties.” In: Kenedi R. editor. Perspectives in Biomedical Engineering: MacMillan, Strathclyde, Glasgow, 1972; 157-160; Maroudas A. “Physical chemistry of articular cartilage and the intervertebral disc.” In: Sokoloff, L., editor, The Joints and Synovial Fluid, Academic Press, New York, 1980, pp. 240-293; Maroudas A. “Variations in the physico-chemical and mechanical properties of human articular cartilage. 1: Physico-chemical properties.” In: Kenedi, R. editor, Perspective in Biomedical Engineering, MacMillan, Strathclyde, 1972, pp. 153-156; Maroudas A, Bayliss M T, Venn M F. “Further studies on the composition of human femoral head cartilage.” Annals of Rheumatic Diseases 1980; 39:514-523; Mizrahi J, Maroudas A, Lanir Y, Ziv I, Webber T J. “The “instantaneous” deformation of cartilage: effects of collagen fiber orientation and osmotic stress.” Biorheology 1986; 23:311-330; Venn M, Maroudas A. “Chemical composition and swelling of normal and osteoarthrotic femoral head cartilage. I. Chemical composition.” Annals of Rheumatic Diseases 1977; 36:121-129].

A variety of bioabsorbable/biodegradable polymers can be used to make the pores in the hydrogel matrix. Examples of biodegradable polymers include degradable polyesters such as polylactic acid, polyglycolic acid or their copolymers, eg. 50:50 PLA:PGA, the degradation profiles of which are well characterized. Examples of other suitable biocompatible, bioabsorbable polymers include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof.

For the purpose of this invention, aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, 5-valerolactone, β-butyrolactone, γ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one 2,5-diketomorpholine, pivalolactone, α,α-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof.

Poly(iminocarbononates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g., PEO/PLA).

Polyalkylene oxalates, for the purpose of this invention, include those described in U.S. Pat. Nos. 4,208,511; 4,141,087, 4,130,639; 4,140,678; 4,105,034; and 4,205,399. Polyphosphazenes, co-, ter- and higher order mixed monomer based polymers made from L-lactide, D,L-lactide, lactic acid, glycolide, glycolic acid, para-dioxanone, trimethylene carbonate and 6-caprolactone are described by Allcock in The Encyclopedia of Polymer Science, Vol. 13, pages 31-41, Wiley Intersciences, John Wiley & Sons, 1988 and by Vandorpe, et. al. in the Handbook of Biodegradable Polymers, edited by Domb, et. al, Hardwood Academic Press, pp. 161-182 (1997).

Polyanhydrides include those derived from diacids of the form HOOC—C₆H₄—O—(CH₂)ₘ—O—C₆H₄—COOH, where “m” is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751, 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et. at, Hardwood Academic Press, pp. 99-118 (1997).

As used herein, the term “glycolide” is understood to include polyglycolic acid. Further, the term “lactide” is understood to include L-lactide, D-lactide, blends thereof, and lactic acid polymers and copolymers.

Exemplary bioabsorbable, biocompatible elastomers include, but are not limited to, elastomeric copolymers of ε-caprolactone and glycolide (including polyglycolic acid) with a mole ratio of ε-caprolactone to glycolide of from about 35 to about 65 to a mole ratio of about 65 to about 35, more preferably from a mole ratio of about 45 to about 55 to a mole ratio of about 35 to about 65; elastomeric copolymers of ε-caprolactone and lactide (including L-lactide, D-lactide, blends thereof, and lactic acid polymers and copolymers) where the mole ratio of ε-caprolactone to lactide is from about 35 to about 65 to a mole ratio of about 65 to about 35 and more preferably from a mole ratio of about 45 to about 55 to a mole ratio of about 30 to about 70 or from a mole ratio of about 95 to about 5 to a mole ratio of about 85 to about 15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40 to about 60 to a mole ratio of about 60 to about 40; elastomeric copolymers of ε-caprolactone and p-dioxanone where the mole ratio of ε-caprolactone to p-dioxanone is from about 30 to about 70 to a mole ratio of about 70 to about 30; elastomeric copolymers of p-dioxanone and trimethylene carbonate where the mole ratio of p-dioxanone to trimethylene carbonate is from about 30 to about 70 to a mole ratio of about 70 to about 30; elastomeric copolymers of trimethylene carbonate and glycolide (including polyglycolic acid) where the mole ratio of trimethylene carbonate to glycolide is from about 30 to about 70 to a mole ratio of about 70 to about 30; elastomeric copolymers of trimethylene carbonate and lactide (including L-lactide, D-lactide, blends thereof, and lactic acid polymers and copolymers) where the mole ratio of trimethylene carbonate to lactide is from about 30 to about 70 to a mole ratio of about 70 to about 30, and blends thereof. Examples of suitable bioabsorbable elastomers are described in U.S. Pat. Nos. 4,045,418; 4,057,537 and 5,468,253.

The biodegradable phase may be admixed with a chemokine specific to the cells of interest. For implantation into cartilage, for example, migration of chondrocytes could be enhanced with a chemoattractant such as IGF-1 or FGF (Chang et al., “Motile chondrocytes from newborn calf: migration properties and synthesis of collagen II,” Osteoarthritis Cartilage, vol. 11, issue 8, p. 603-12, August 2003). For implantation into bone, chemoattractants such as BMP2 could encourage osteoblast migration (Lind et al, Bone, vol. 18, issue 1, p. 53-57, January 1996). For the purposes of filling a bony defect and cartilage defect combined (in the case of an osteochondral defect) a layered plug with different chemoattractants in each layer would be utilized. In a preferred embodiment, the biodegradable phase will contain specific chemokines to stimulate cell migration, adhesion, proliferation, and extracellular-matrix synthesis. In addition, in a preferred embodiment, the non-degradable phase will contain specific chemokines to stimulate cell migration, adhesion, proliferation, and extracellular-matrix synthesis.

The compositions according to the invention are formulated by the admixture of a biodegradable polymer and a non-biodegradable hydrogel to form a dispersion of microspheres of the biodegradable polymer in a matrix of the non-biodegradable hydrogel. The microspheres can be preformed or generated in situ. For example, preformed microspheres in a non-aqueous solvent are combined with an aqueous solution of hydrogel-forming polymer to form an emulsion. Alternatively, the biodegradable polymer (or monomers thereof) and optional additional ingredients are dissolved in a solvent to be dispersed in an aqueous solution of hydrogel-forming polymer to form an oil-in-water emulsion with formation of microspheres in situ.

Suitable solvents for admixing the components of the compositions according to the invention depend on the nature of the biodegradable polymer and non-biodegradable hydrogel, and typically may include water, saline solution, aqueous buffer, dichloromethane, acetone, ethyl acetate, acetonitrile, methanol, ethanol, isopropanol, butanol, amyl alcohol, ethylene glycol, diethylene glycol, hexanes, dodecane, toluene, cyclohexanone, diethyl ether, tetrahydrofuran, ethyl lactate, fluorocarbons, alcohols, alkanes, pyridine, dimethylformamide, benzene, chloroform, light petroleum, carbon tetrachloride, dichloroethane, dioxane, carbon disulphide, dimethyl sulphoxide, mineral oil, natural oils, and the like.

Once mixed, compositions according to the invention can be poured into a mold, the geometry of which is dictated by the defect size and shape to be treated. The hydrogel matrix can also be shaped using injection molding techniques, the geometry of the mold thus dictating the final shape of the implant. Using MRI or CT images of the defect to obtain patient-specific geometry, the implants can be molded to match the shape of the defect. Alternatively, more simple shapes can be molded, e.g. cylinders, cones, or ovoids, and the defect can be machined to match the plug.

By the term “biocompatible” is meant a composition that is suitable for implant into living tissue. The term “microspheres” includes encapsulated material, including microparticles, but does not require absolute spherical shape. Thus, the term “microspheres” also encompasses, for example, micro-ovoids and related structures such as microtubules and channels resulting from overlapping microspheres. When spherical, the microspheres will preferably have an average radius of about 10 to as much as about 500 microns. A “biodegradable” component is one that, when exposed to in vivo conditions, will decompose over time and be metabolized or removed from the tissue. Suitable time ranges for decomposition include one week to two years. A non-biodegradable component is one that is stable over time, with a minimal amount of decomposition, such that the component maintains its structural integrity and is substantially the same after a set period of time, such as 2 years, 5 years, or 10 years. It will be recognized that the microtubules, channels, or pores resulting from the degradation of the biodegradable component should be in an “open pore” formation. In other words, the structures resulting from degradation of the biodegradable component are preferably interconnected to allow for ingress of biological tissue into the structural voids, while maintaining sufficient non-biodegradable hydrogel matrix for long-term structural integrity.

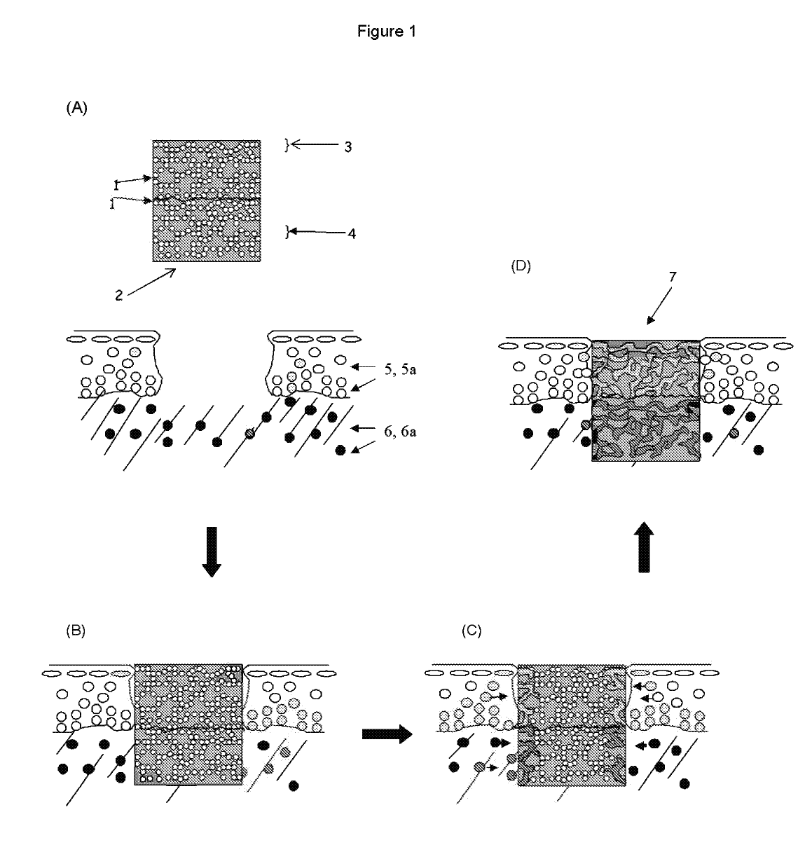

An application of the invention, specific to the aim of repairing focal osteochondral defects, is illustrated in FIG. 1 In part A of FIG. 1, the specimen is a bi-layered structure where the degradable phase takes the form of micron-sized spheres (1), tubes or similar geometries that would create pores upon degradation distributed throughout a non-degradable porous matrix (2). The chemoattractant incorporated into the degradable spheres in the upper part of the plug is that which attracts chondrocytes (for example IGF-1), while the chemoattractant in the lower portion of the plug is that which attracts osteoblasts or other cell types from the bone (for example, BMP-2). The upper half of the plug (3) is a cartilage implant, while the lower half of the plug (4) is a bone implant. An osteochondral defect near cartilage (5) containing chondrocytes (5a) and bone (6) containing osteoblasts or other cell types (6a) is shown below the implant in part A of FIG. 1. When the semi-degradable microsphere-seeded hydrogel implant is placed into the osteochondral defect as in part B of FIG. 1, the microspheres on the periphery will start to degrade, thus releasing chemoattractant. Cells are attracted and begin to migrate into the channels, chondrocytes into the upper layer and osteoblasts or other cell types from the bone into the bottom layer as shown in part C of FIG. 1. Degradation continues, more chondrocytes migrate into the pores of the hydrogel, generate extracellular matrix, and help to integrate the interface, ultimately resulting in a composite tissue-hydrogel plug (7), as shown in part D of FIG. 1.

In addition to the pores that will open up as the microspheres degrade, other pores or channels may also exist throughout the non-degradable matrix as a result of the technique used to prepare the network. By manufacturing hydrogels according to Example 3 below, a porous fluid-filled matrix is produced. When subjected to a compressive force, the fluid from within the structure permeates through the matrix and escapes through the surface of the material. This feature can allow the material to express fluid through its surface, which when articulated against native articular cartilage, can act to lubricate and separate the opposing articulating surfaces, much in the way that articular cartilage functions in the normal joint (i.e., cartilage-cartilage contact).

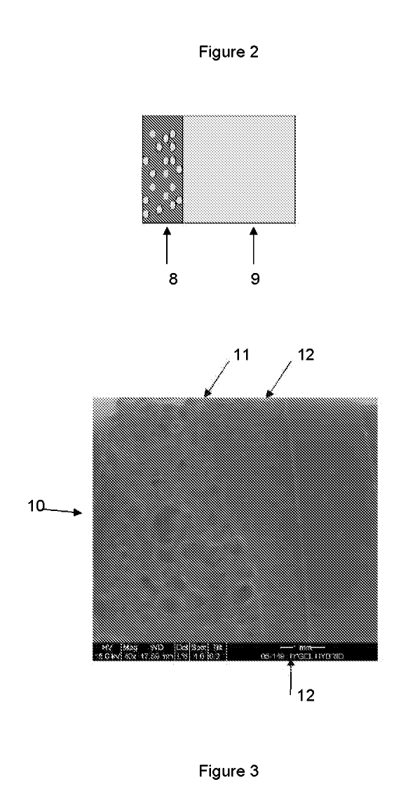

Movement of fluid through the hydrogel's interior and expression out of the hydrogel's surface makes these materials uniquely suited for use in the repair or replacement of the articular surface of articulating (movable or diarthrodial) joints—either for meniscal replacement or articular cartilage replacement. For example, the construct could be manufactured with inhomogeneous and anisotropic properties, such as where the porous construct could be manufactured with a more compressible lubricating layer (surface) on top of a stiffer or stronger base layer (bottom), as illustrated in FIG. 2. According to the layered structure of FIG. 2, a surface hydrogel layer with pre-existing pores (8) and a lower hydrogel layer (9) would be of a different formulation. The manufacturing of this layered structure would involve first subjecting the base hydrogel layer (with or without solvent-induced pores) to 2 or 3 freeze-thaw cycles, and then pouring a solvent-induced porous hydrogel onto the base layer surface and freeze-thawing the entire construct for a further 3-4 cycles. This would produce a stiffer base layer covered with a more compressible porous surface layer. As shown in FIG. 3, a hydrogel material (with solvent-induced pores) (10) is adjacent to a hydrogel matrix without solvent-induced pores (12) with a well-integrated interface (11) between the hydrogel layers. Such a layered structure can be combined with the features of degradation-induced pores according to FIG. 1 to provide an implant that integrates into the tissue over time and expresses lubricating fluid through its surface. This structure could also be adapted to allow for peripheral integration.

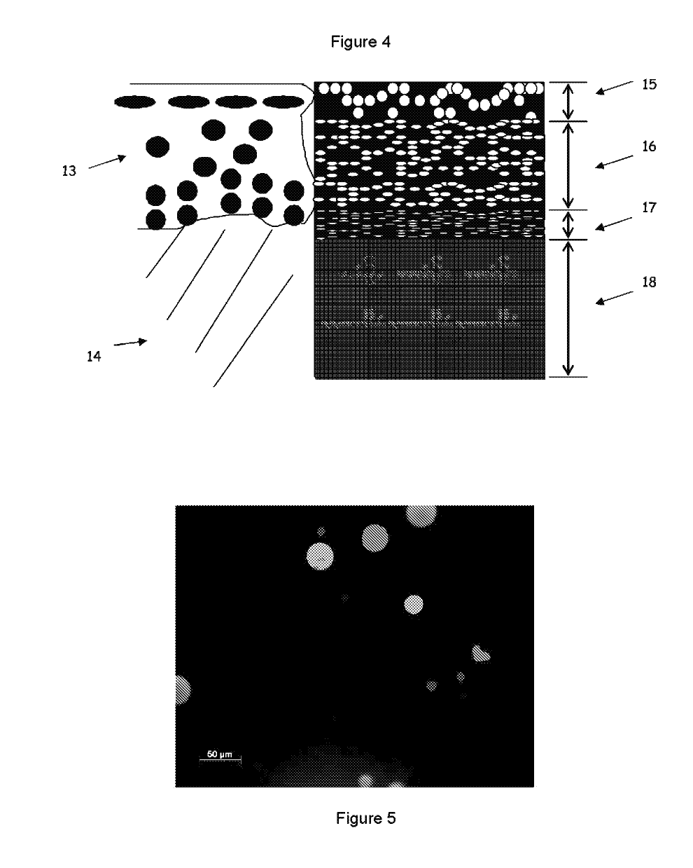

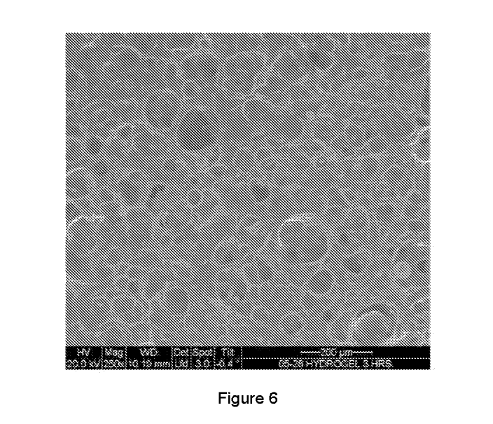

Other hydrogel constructs could be manufactured with multiple layers (two or more), each layer having different combinations of inhomogeneous and anisotropic properties, or even a construct having continually varying properties throughout the construct. These constructs could be manufactured to specifically match the functional properties of the desired tissue to be repaired or replaced. In the case of articular cartilage, an inhomogeneous and anisotropic construct could be manufactured with multiple layers or continuously varying regions, starting from the articular surface to the underlying bone, to duplicate the functional properties of the superficial, middle and deep zones, the cartilage-subchondral bone interface (calcified cartilage, tidemark), and the underlying bone [Arnoczky S, Torzilli P. “The biology of cartilage.” In: Hunter L F, Funke F J, editors. Rehabilitation of the Injured Knee: C V Mosby; 1984. p 148-209; Torzilli P. “The lubrication of human joints: a review.” In: Fleming D F, B N, editor. Handbook of Engineering in Medicine and Biology. Ohio: CRC Press; 1976 p 225-251]. For example, the hydrogel construct could be manufactured with (1) a lower compressive modulus and higher permeability and porosity at the surface or uppermost layer (duplicating the superficial zone) that would articulate with the opposing side of the joint, (2) have increasing compressive modulus and decreasing permeability and porosity with increasing distance from the articular surface (duplicating the middle and deep zones), which would extent to a depth equivalent to the cartilage-subchondral bone interface, (3) have another hydrogel construct at the cartilage-bone interface to separate the cartilage and bone, such as a semi-permeable or impermeable hydrogel (duplicating the calcified cartilage), and (4) have a much stiffer hydrogel within the underlying bone that would be more like the surrounding bone into which it would be inserted. A hydrogel composite that was manufactured with this specific composition of multiple layers or continuously varying regions could duplicate the functional properties of articular cartilage, which vary continuously with depth from the articular surface to the subchondral bone, see FIG. 4 [Charlebois M, McKee M D, Buschmann M D. “Nonlinear tensile properties of bovine articular cartilage and their variation with age and depth.” Journal of Biomechanical Engineering 2004; 126:129-137; Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Chen S S, Falcovitz Y H, Schneiderman R, Maroudas A, Sah R L., “Depth-dependent compressive properties of normal aged human femoral head articular cartilage: relationship to fixed charge density.” Osteoarthritis and Cartilage 2001; 9(5):561-569; O'Connor P, Orford C R, Gardner D L. “Differential response to compressive loads of zones of canine hyaline articular cartilage: micromechanical, light and electron microscopic studies.” Annuals of Rheumatic Diseases 1988; 47(5):414-420; Schinagl R M, Gurskis D, Chen A C, Sah R L. “Depth-dependent confined compression modulus of full-thickness bovine articular cartilage.” Journal of Orthopaedic Research 1997; 15(4):499-506; Torzilli P. “Measurement of the compressive properties of thin cartilage slices: evaluating tissue inhomogeneity.” In: Maroudas A K, K, editor. Cartilage Methods. New York: Academic Press; 1990. p 304-308; Wang C C, Hung C T, Mow V C. “An analysis of the effects of depth-dependent aggregate modulus on articular cartilage stress-relaxation behavior in compression.” Journal of Biomechanics 2001; 34(1):75-84]. FIG. 4 is a diagram showing a multilayered hydrogel where porosity, permeability and modulus vary through the depth to more closely mimic the mechanical behavior of the adjacent cartilage (13) and bone (14). In FIG. 4, region (15) has low compressive modulus and high permeability and high porosity; region (16) has high compressive modulus, low permeability, and low porosity; region (17) resembles a membrane that is semi-permeable or impermeable; and region (18) is a stiffer, less porous hydrogel. For instance, the hydrogel implant would have one or more properties like articular cartilage such as an equilibrium aggregate modulus (Ha) which increases by almost two orders of magnitude (100 times) from the articular surface to the deep zone, 0.01 MPa to 10 MPa [Chahine N O, Wang C C, Hung C T, Ateshian G A. “Anisotropic strain-dependent material properties of bovine articular cartilage in the transitional range from tension to compression.” Journal of Biomechanics 2004; 37:1251-1261; Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12, Chen S S, Falcovitz Y H, Schneiderman R, Maroudas A, Sah R L. “Depth-dependent compressive properties of normal aged human femoral head articular cartilage: relationship to fixed charge density.” Osteoarthritis and Cartilage 2001; 9(5):561-569; O'Connor P, Orford C R, Gardner D L. “Differential response to compressive loads of zones of canine hyaline articular cartilage: micro mechanical, light and electron microscopic studies.” Annuals of Rheumatic Diseases 1988; 47(5):414-420; Schinagl R M, Gurskis D, Chen A C, Sah R L. “Depth-dependent confined compression modulus of full-thickness bovine articular cartilage.” Journal of Orthopaedic Research 1997; 15(4):499-506; Torzilli P. “Measurement of the compressive properties of thin cartilage slices: evaluating tissue inhomogeneity.” In: Maroudas A K, K, editor. Cartilage Methods. New York: Academic Press, 1990. p 304-308; Wang C C, Hung C T, Mow V C. “An analysis of the effects of depth-dependent aggregate modulus on articular cartilage stress-relaxation behavior in compression.” Journal of Biomechanics 2001; 34(1):75-84]; Young's modulus (E) which increases from 0.10 MPa to 2.0 MPa from the articular surface to the deep zone [Brocklehurst R, Bayliss M, Maroudas A, Coysh H, Freeman M, Revell P A. “The composition of normal and osteoarthritic articular cartilage from human knee joints.” Journal Bone and Joint Surgery 1984, 66A:95-106; Charlebois M, McKee M D, Buschmann M D. “Nonlinear tensile properties of bovine articular cartilage and their variation with age and depth.” Journal of Biomechanical Engineering 2004; 126:129-13; Chen S S, Falcovitz Y H, Schneiderman R, Maroudas A, Sah R L. “Depth-dependent compressive properties of normal aged human femoral head articular cartilage: relationship to fixed charge density.” Osteoarthritis and Cartilage 2001; 9(5):561-569; Wang C C, Hung C T, Mow V C. “An analysis of the effects of depth-dependent aggregate modulus on articular cartilage stress-relaxation behavior in compression.” Journal of Biomechanics 2001; 34(1):75-84]; Instantaneous modulus (E at 2 sec) increases from 1.0 MPa to 20.0 MPa from the articular surface to the deep zone [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Chen S S, Falcovitz Y H, Schneiderman R, Maroudas A, Sah R L. “Depth-dependent compressive properties of normal aged human femoral head articular cartilage: relationship to fixed charge density.”. Osteoarthritis and Cartilage 2001; 9(5):561-569; Wang C C, Hung C T, Mow V C. “An analysis of the effects of depth-dependent aggregate modulus on articular cartilage stress-relaxation behavior in compression.” Journal of Biomechanics 2001; 34(1):75-84]; Permeability (k) decreases from 2.0×10⁻¹⁵ m⁴/Ns to 0.1×10⁻¹⁵ m⁴/Ns from the articular surface to the deep zone [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12]; Bulk modulus increases from 0.1 MPa to 3.0 MPa from the articular surface to the deep zone [Chen A C, Bae W C, Schinagl R M, Sah R L. “Depth- and strain-dependent mechanical and electromechanical properties of full-thickness bovine articular cartilage in confined compression.” Journal of Biomechanics 2001; 34(1):1-12; Chen S S, Falcovitz Y H, Schneiderman R, Maroudas A, Sah R L. “Depth-dependent compressive properties of normal aged human femoral head articular cartilage: relationship to fixed charge density.” Osteoarthritis and Cartilage 2001; 9(5):561-569; Maroudas A. “Physical chemistry of articular cartilage and the intervertebral disc.” In: Sokoloff, L., editor, The Joints and Synovial Fluid, Academic Press, New York, 1980, pp. 240-293]; Poisson's ratio (v) increases from 0.01 to 0.50 from the articular surface to the deep zone [Mizrahi J, Maroudas A, Lanir Y, Ziv I, Webber T J. “The “instantaneous” deformation of cartilage: effects of collagen fiber orientation and osmotic stress.” Biorheology 1986; 23:311-330, Wang C C, Hung C T, Mow V C. “An analysis of the effects of depth-dependent aggregate modulus on articular cartilage stress-relaxation behavior in compression.” Journal of Biomechanics 2001; 34(1):75-84]; dynamic modulus (E_{dyn} for 0.005 to 1 Hz) increases from 0.1 MPa to 30 MPa from the articular surface to the deep zone [O'Connor P, Orford C R, Gardner D L. “Differential response to compressive loads of zones of canine hyaline articular cartilage: micro mechanical, light and electron microscopic studies.” Annuals of Rheumatic Diseases 1988; 47(5):414-420; Torzilli P. “Measurement of the compressive properties of thin cartilage slices: evaluating tissue inhomogeneity.” In: Maroudas A K, K, editor. Cartilage Methods. New York: Academic Press; 1990. p 304-308]; and water content increases from 85% to 70% [Maroudas A. “Variations in the physico-chemical and mechanical properties of human articular cartilage. 1: Physico-chemical properties.” In: Kenedi, R. editor, Perspective in Biomedical Engineering, MacMillan, Strathclyde, 1972, pp. 153-156; Maroudas A, Bayliss M T, Venn M F. “Further studies on the composition of human femoral head cartilage.” Annals of Rheumatic Diseases 1980; 39:514-523; Torzilli P, Askari E, Jenkins J. “Water content and solute diffusion properties of articular cartilage.” In: Mow V R, A; Woo, S L-Y, editor. Biomehanics of Diarthroidial Joints. New York: Springer-Verlag; 1990. p 363-390]. A hydrogel construct manufactured with these depth-varying mechanical and material properties would duplicate the functional properties of articular cartilage.

For example, when the hydrogel construct is placed into an osteochondral defect and compressed when the joint is loaded, the less stiff and more permeable uppermost region of the hydrogel construct would be easily compressed and exude the interstitial fluid within this region through the hydrogel surface, which would act to separate and lubricate the joint surfaces [Arnoczky S, Torzilli P. “The biology of cartilage.” In: Hunter L F, Funke F J, editors. Rehabilitation of the Injured Knee: CV Mosby; 1984. p 148-209; Torzilli P. “The lubrication of human joints: a review.” In: Fleming D F, B N, editor. Handbook of Engineering in Medicine and Biology. Ohio: CRC Press; 1976. p 225-251]. The stiffer and less permeable regions below would resist the load and deform less, keeping the articulating surfaces and interface of the hydrogel construct and adjacent (surrounding) articular cartilage aligned.

### EXAMPLES

In order that the present invention may be more readily understood, specific non-limiting examples are shown below.

In one possible formulation the non-biodegradable component is manufactured from polyvinyl alcohol (PVA), and the biodegradable component is manufactured from poly lactic glycolic acid (PLGA). Three methods of manufacture are presented to produce a composite degradable and hydrophilic implant, where a chemoattractant is incorporated into the degradable spheres. Alternatively, the chemoattractant can take the form of fibers, such as produced using electrospinning techniques.

### Example 1

### Microspheres Manufactured and Dispersed in a Poly(Vinyl Alcohol) Solution

Dilute PVA aqueous solution was prepared by dissolving 88% hydrolyzed PVA in deionized water at 83° C. for 2 hours to form an external aqueous phase. PLGA with a lactic-to-glycolic ratio of 50:50 (Medisorb® 5050DL 3.5A, I.V. 0.40 dl/g) was dissolved in organic solvent (dichloromethane/acetone), and suspended in sterile PBS or the chemoattractant of interest to form an internal aqueous phase.

Ten ml of each solution was dispersed in continuous phase and homogenized for 5 minutes to produce an oil-in-water emulsion. The microsphere size was controlled by the speed of stirring. Typical diameters range from 2 μm to 100 μm. The emulsion was transferred onto a magnetic stir plate for four hours to remove dichloromethane and to harden the microparticles. The microparticles were collected through a centrifuge at 15,000 rpm at 10° C. for 45 minutes. The pellets were frozen at −80° C. and then freeze-dried overnight. The freeze-dried PLGA microparticles were dispersed in deionized water and sonicated for 30 seconds to make 50 wt % particles suspension with an even distribution of microparticles throughout the polymer solution. Optionally, a surfactant such as Tween can be added to help disperse the particles.

The mixture was poured into a mold, the geometry of which was dictated by the defect size and shape, and subjected to 5 cycles of freezing each for 23 hours at −20° C. with two hours of thawing at room temperature, about 25° C., in between each freeze cycle. FIG. 5 is a photograph of microspheres made according to Example 1.

Compressive Young's Modulus (E) was approximately 0.1 MPa Compressive Aggregate Modulus (Ha) was approximately 0.6 MPa and Dynamic Modulus (E_{dyn}) was approximately 4 MPa.

### Example 2

### PLGA Microspheres Suspended as an Oil-in-Water Emulsion in PVA Matrix

Dilute (10 wt %) PVA aqueous solutions were prepared by dissolving PVA (Elvanol 71-30; Mw˜96,000) from DuPont (Wilmington, Del.) in deionized water at 83° C. for 2 hours. PLGA (Medisorb® 5050DL 3.5A) supplied by Alkermes, Inc. (Cincinnati, Ohio) was dissolved in dichloromethane (DCM) where the amount of DCM used was 10 times the weight of PLGA, and the mixture was sonicated using a Branson (Danbury, Conn.) Bransonic® 1510 ultrasonic cleaner until dissolved. The dissolved PLGA was added to the PVA and the solution was tented with tinfoil and magnetically stirred at 300 rpm for 10 minutes. The mix was poured into a mold and manually subjected to 5 cycles of freezing at −20° C., each for 23 hours, with 1 hour of thawing at 25° C. in between each freeze cycle.

Supplemental information for EXAMPLE 2.

Method of manufacture and analysis: The amount of PLGA solution added was varied as a weight percent of PVA to produce the following groups: 10%, 20%, 50%, and 75% PLGA. The emulsion was poured into wells of a 24-well polystyrene plate, sealed with parafilm and subjected to five cycles of 23 hours of freezing (−20° C.) followed by 1 hour of thawing at room temperature (25° C.) in a MicroClimate™ chamber (Cincinnati Sub-Zero, Cincinnati, Ohio).

Morphology: The surface of all samples for morphological analysis (n=5 per group) was removed using a freezing stage microtome. Samples were placed in an environmental chamber of a scanning electron microscope (FEI Philips, Hillsboro, Oreg.). Two images, one at 250× and one at 500× magnification, of each of the 10%, 20%, 50% and 75% PLGA samples were imported into J-image (National Institutes of Health, Bethesda, Md.). Using the 500× images, pores with diameters greater than 10 microns (limit of resolution of the system) were identified and the diameters recorded. Similarly, microspheres with diameters greater than 7 microns were identified and diameters measured. Percent porosity was calculated from the 250× images, as the total pore area as a percent of the field of view.

Mechanical Testing: Hydrogels (n=10 per group) were cored using a 5 mm diameter biopsy punch and sliced on a freezing stage microtome to ensure flat parallel surfaces and a thickness of 2-3 mm. Mechanical testing was performed on a custom-made test apparatus, the Compression Computer Automated Soft Tissue Test System (CCASTTS) [Torzilli, P., Depres, A., McKibben, D., Chan, B., Co, F., Wenz, J., and Carr, J., 1988. A device for measuring the compressive properties of thin specimens. ASME Advances in Bioengineering, BED-8, 179-180]. Samples were confined in a 5 mm diameter stainless steel well and subjected to either a stress relaxation test (n=5) or a creep test (n=5). Samples were loaded via a porous 5 mm diameter brass filter (25-35 μm porosity).

For the stress relaxation test the sample was rapidly displaced (compressed) at a rate of 22 μm/s up to 5% strain. The displacement was held constant and the change in load recorded until equilibrium, or 1800 seconds, was reached. This was repeated for each of five steps, up to a total strain of 25%. Stress vs. strain was plotted for the loading phase of each step. An exponential function was curve fit to the stress vs. strain data for each step. The slope at the peak strain for each loading step was computed as the Dynamic Modulus (E_{dyn}). Dynamic modulus vs. strain was plotted as a function of PLGA content.

For the creep test, the confined hydrogel samples were rapidly loaded (compressed) at a rate of 150 μm/s, to a load of 50 g. Thereafter the load was held constant and samples were allowed to creep (deform) for one hour. Throughout testing the hydrogel displacement was recorded. The creep test was analyzed using the biphasic theory [Mow, V., Kuei, S., Lai, W., and Armstrong, C., 1980. Biphasic creep and stress relaxation of articular cartilage in compression: Theory and experiments. Journal of Biomechanical Engineering, 102, 73-84] to calculate the Aggregate Modulus, Ha, and permeability for each test site.

Cellular Response: 50% PLGA and 75% PLGA samples (n=5 per group) were sliced to a thickness of 3 mm. Hydrogel slices were placed into a 12-well plate and soaked in culture medium overnight; after which medium was removed in preparation for cell seeding. The culture medium consisted of Dulbecco's Modification of Eagle's Medium (Mediatech, Inc., Herndon Park, Va.), 10 vol % fetal bovine serum, and 1 vol % antibiotic-antimicotic (Gibco, Invitrogen Corporation, Grand Island, N.Y.). Chondrocytes were isolated from the articular cartilage of the weight-bearing areas of adult bovine femoral condyles. Cells were seeded onto the top surface of the hydrogel constructs in a spot volume of 50 μl at a density of 1×10⁶ cells/50 μl (˜6000 cells/mm). Samples were placed in an incubator for ninety minutes, after which medium was added. Medium was replaced every 2-3 days. At 2 weeks and 4 weeks, samples were placed in fixative, embedded in paraffin, sliced to 7 μm, and stained with Alcian Blue and Kernechtrot.

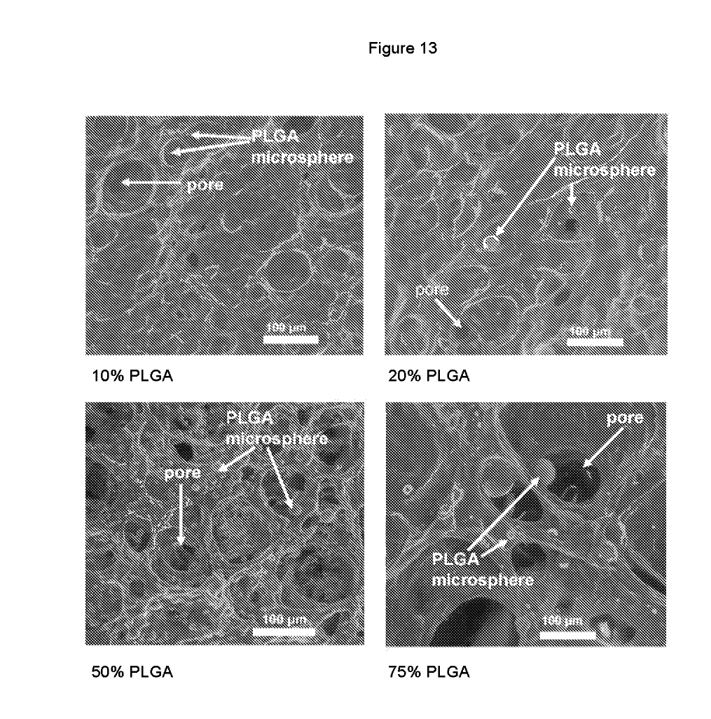

FIG. 13 shows SEM images of 10%, 20%, 50% & 75% PLGA hydrogels. In these representative pictures it can be seen by varying PLGA content, average pore diameter, percent porosity, and microsphere diameter can be controlled.

TABLE 1
Hydrogel Porosity (%) and Pore Diameter, and PLGA
Microsphere Diameter (mean ± standard deviation)

| % PLGA | % Porosity | Sphere Diameter (μm) | Pore Diameter (μm) |
|---|---|---|---|
| 10 | 8 ± 3 | 8.5 ± 2.1 | 29 ± 21 |
| 20 | 17 ± 5 | 10.8 ± 3.9 | 35 ± 22 |
| 50 | 49 ± 20 | 25.4 ± 25.5 | 95 ± 82 |
| 75 | 54 ± 19 | 33.7 ± 34.7 | 111 ± 85 |

TABLE 2
Compressive Aggregate Modulus and Permeability
(mean ± standard deviation)

| | Ave. Aggregate | | Ave. Dynamic |
|---|---|---|---|
| PLGA % | Modulus, Ha | Ave. Permeability, k | Modulus |
| 10 | 0.102 ± 0.013 | 2.74 ± 0.56 | 21.4 ± 7.8 |
| 20 | 0.087 ± 0.0075 | 1.53 ± 0.68 | 4.7 ± 2.0 |
| 50 | 0.101 ± 0.015 | 1.39 ± 0.39 | 9.2 ± 5.0 |
| 75 | 0.097 ± 0.0087 | 4.32 ± 1.05 | 1.67 ± 2.93 |

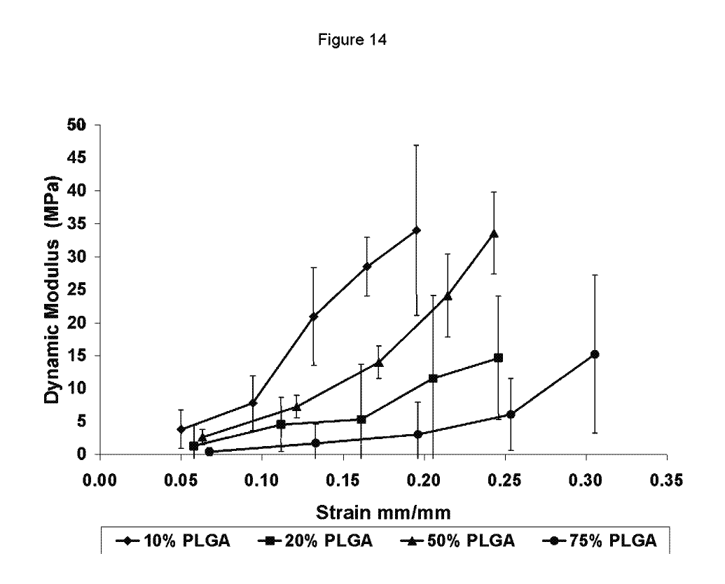

FIG. 14 shows the Dynamic Modulus vs. strain as a function of percent PLGA. The general trend demonstrated was such that dynamic modulus increased as percent PLGA decreased.

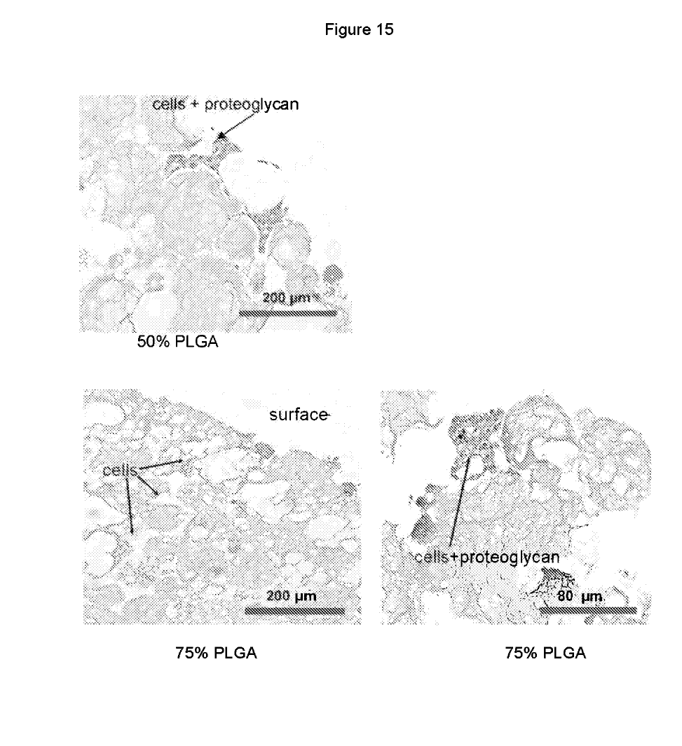

FIG. 15 shows a 2-week, chondrocyte-seeded hydrogel. Top row is 50% PLGA; Bottom row images are of 75% PLGA samples. Blue stains for proteoglycan.

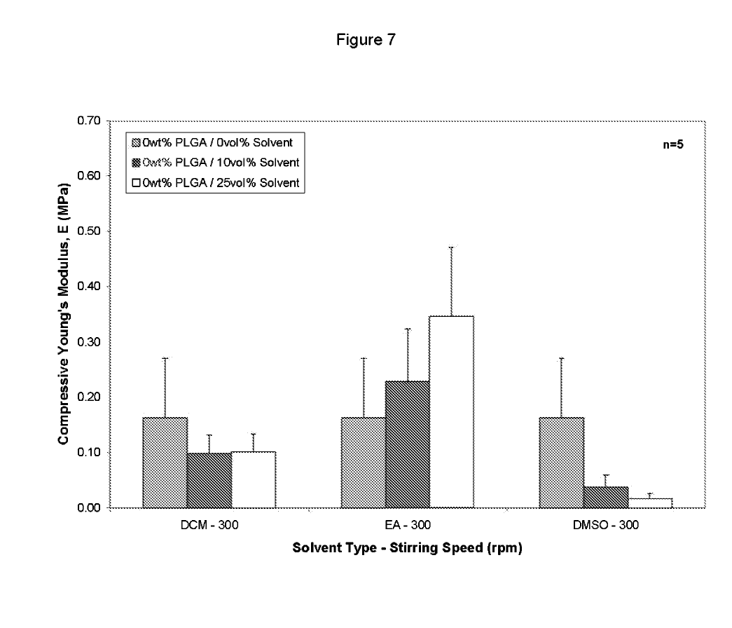

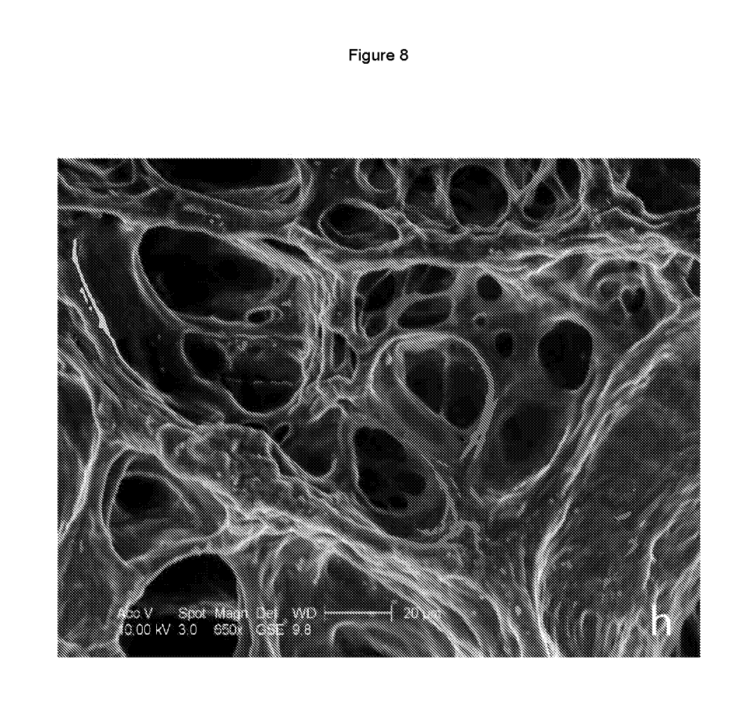

Dichloromethane, ethyl acetate, or dimethyl sulfoxide was added to 25 ml of the PVA solution in the amounts of 0, 10, and 25 vol %. The emulsions were stirred at 300 rpm for ten minutes using a magnetic stirrer or homogenized at 300 rpm or 1000 rpm for ten minutes, using a Brinkman probe homogenizer (Model: PT3100) (Westbury, N.Y.), and then poured into a mold and exposed to repetitive freeze-thaw cycles. The Compressive Young's Modulus of the hydrogels, FIG. 7 (unconfined, uniaxial compression) ranged from about 0.1 to 0.4 MPa. FIG. 8 shows a cross section of the transverse plane of a sample made with 10 vol % ethyl acetate and stirred at 300 rpm, imaged in its hydrated state using environmental scanning electron microscopy. The pores are between 10 and 50 μm.

### Example 3

### Water-in-Oil-in-Water Solvent Evaporation/Extraction Method (IGF Chemoattractant)

A 0.15 ml internal aqueous solution (0.0016 M Citric Acid, 5% w/v human serum albumin (HSA), 2.5 mg additional HSA, and 1 mg chemoattractant (insulin-like growth factor—IGF)) was added to 2 ml of an organic solution (1.5 ml methylene chloride, 0.5 ml acetone) containing dissolved poly(lactic acid) (50 mg). The combined solution was sonicated for 15 seconds in a glass vial, then added to 30 ml of a 5% w/v external aqueous solution of PVA to achieve a multiple emulsion and stirred at 500 rpm for 1 minute. The combined PVA solution was then added to 400 ml solution of 10% PVA-PVP in water and stirred at 500 rpm for 25 minutes to remove the organic solvent, where the 10% PVA-PVP solution was prepared by dissolving 99% by weight polyvinyl alcohol (PVA, Elvanol™ Grade 71-30, DuPont, Wlimington, Del.) and 1% polyvinyl pyrrolidone (PVP, MW=40,000, Sigma Aldrich, St. Louis, Mo.) in deionized water at 90° C. for 24 hours to yield a 10% polymer solution. The solutions were poured into a mold and exposed to repetitive freeze-thaw cycles.

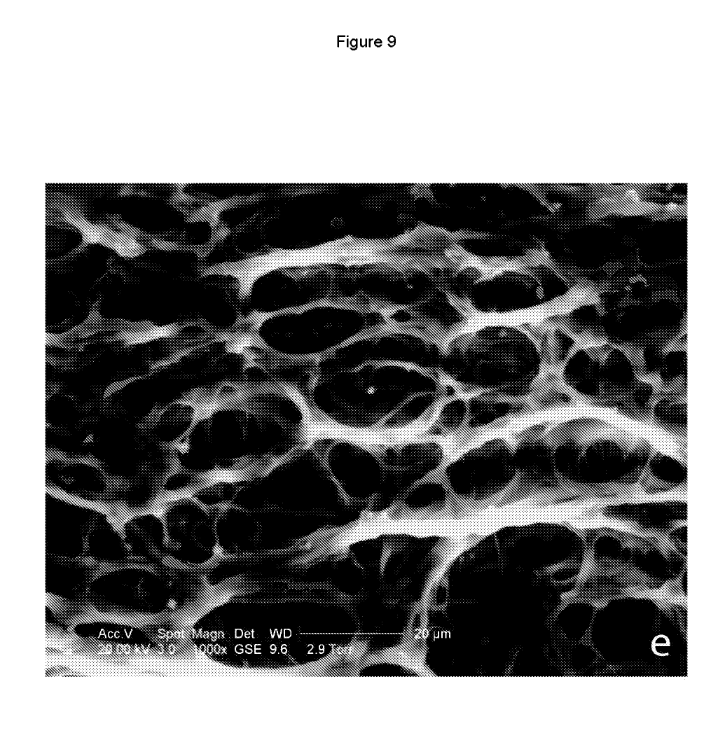

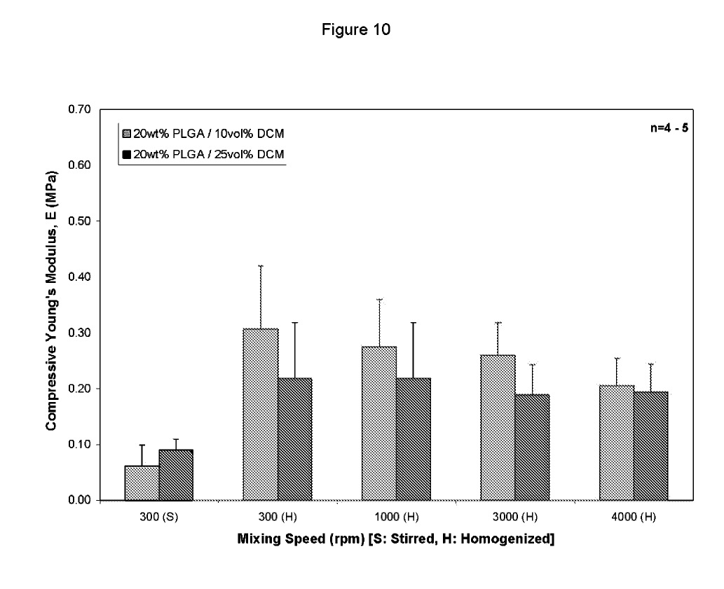

Microparticles were also created in the hydrogels by adding an internal aqueous phase in the amount of 12.5 vol % to a solution of PLGA in dichloromethane or ethyl acetate. This emulsion was sonicated for 5 minutes and then added to the PVA-PVP solution, forming a double emulsion, so that the ratio of PLGA to PVA-PVP was 1:3, and the amount of dichloromethane was 10 or 25 vol %. The mixture was stirred at 300 rpm or homogenized at speeds between 300 and 4000 rpm. FIG. 9 shows a sample made with 2 wt % PLGA and 25 vol % dichloromethane. The compressive Young's modulus of these hydrogels ranged from 0.05 to 0.3 MPa as shown in FIG. 10.

### Example 4

### PLGA Microparticles Collected and Suspended in PVA Matrix

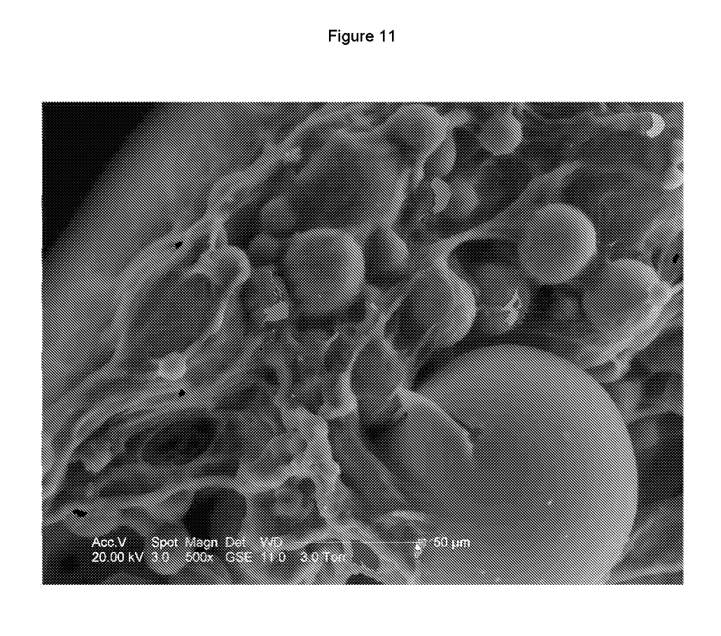

Microspheres were fabricated by adding 2 ml internal aqueous solution (1 mg chemoattractant-insulin) 15 ml of an organic solution (10 ml dichloromethane, 5 ml acetone) containing dissolved poly(lactic-co-glycolic acid) (0.5 g). This emulsion was stirred at 300 rpm for 30 minutes and then added to 150 ml of 2% PVA (Polysciences 88% hydrolyzed, Mw˜25,000) to achieve a multiple emulsion, which was then stirred at 400 rpm for 4 hours to remove the organic solvent. The mixture was centrifuged at 3500 rpm for 25 minutes to collect the microparticles, which were then added to the 25 ml of 10% PVA solution and stirred at 300 rpm for 10 minutes. This mixture was added to a mold and exposed to repeated cycles of freezing and thawing. The compressive Young's modulus of these hydrogels was about 0.4 MPa. FIG. 11 shows a sample made with 5 wt % microparticles.

### Example 5

### Microparticles Collected and Suspended in PVA Matrix with Addition Organic Solvent as Pore-Forming Agent

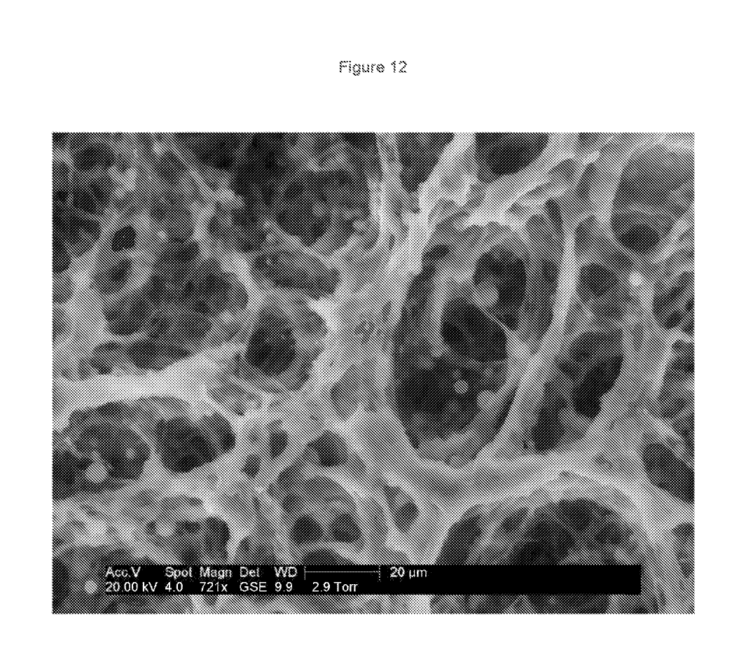

Microparticles were fabricated and collected according to the composition in Example 4, and added to 25 ml of 10% PVA solution. Various organic solvents, such as dichloromethane, ethyl acetate, acetone, ethanol and isopropanol, were added to this mixture to create an emulsion, which was stirred at 300 rpm for 10 minutes. This mixture was added to a mold and exposed to repeated cycles of freezing and thawing. FIG. 12 shows a sample made with 2 wt % microparticles and 25 vol % dichloromethane.

From the above description, one can ascertain the essential characteristics of the present invention and, without departing from the spirit and scope thereof, can make various changes and modifications of the invention to adapt it to various uses and conditions.

We claim:

1. A biocompatible composition for treating, repairing, or replacing biological tissue comprising microspheres of a biodegradable polymer dispersed in a matrix of a non-biodegradable hydrogel, wherein the composition is formulated to have compatible mechanical properties and physical functionality with the biological tissue.

2. A composition according to claim 1, wherein upon biodegradation, the microspheres form open-celled pores.

3. A composition according to claim 1 where the composition has pre-existing pores throughout its structure.

4. A composition according to claim 1, wherein the non-biodegradable hydrogel is polyvinyl alcohol.

5. A composition according to claim 1, wherein the non-biodegradable hydrogel is polyvinyl alcohol and polyvinylpyrrolidone.

6. A composition according to claim 1, wherein the biodegradable polymer is poly lactic glycolic acid.

7. A composition according to claim 6, wherein the mole ratio of lactic to glycolic acid is about 50:50.

8. A composition according to claim 1, wherein the hydrogel further comprises a chemoattractant, chemokine, cytokine, adhesion molecules, or mixtures thereof.

9. A composition according to claim 1, wherein the microspheres further comprise a chemoattractant, chemokine, cytokine, adhesion molecules, or mixtures thereof.

10. A composition according to claim 9, wherein the chemoattractant is selected from the group consisting of IGF-1, FGF, BMP2, and mixtures thereof.

11. A composition according to claim 9, wherein the chemoattractant promotes the biosynthesis of extracellular-matrix.

12. A composition according to claim 9, wherein the type and amount of chemokine is compatable with the biological tissue.

13. A composition according to claim 1, wherein the microspheres have an average diameter between about 10 and about 100 microns.

14. A composition according to claim 1, wherein the composition is molded in the shape of a biological tissue defect.

15. A composition according to claim 1, wherein the composition is molded in the shape of a cylinder, cone, or ovoid for insertion into a biological tissue defect.

16. A composition according to claim 1, wherein the biological tissue is selected from the group consisting of cartilage, bone, bladder, liver, kidney, and mixtures thereof.

17. A composition according to claim 1, wherein the microspheres are unevenly dispersed in the matrix to form pores, avenues, or channels upon degradation.

18. A composition according to claim 17, wherein the pores, avenues, or channels formed upon degradation are sufficiently large to permit cellular migration into the remaining matrix of non-biodegradable hydrogel.

19. A composition according to claim 1, wherein the microspheres of biodegradable polymer are selected to degrade in vivo at a rate such that mechanical and functional characteristics of the implanted composition are maintained by the simultaneous ingrowth of biological material.

20. The composition according to claim 1, wherein the composition contains two or more discreet layers, wherein each layer has compatible mechanical properties and physical functionality for a distinct biological tissue.

21. The composition according to claim 20, wherein the composition has two layers, and further wherein one layer is compatible with bone and one layer is compatible with cartilage.

22. A method of making a composition according to claim 1, comprising forming microspheres containing a chemokine, dispersing the microspheres in a hydrogel matrix, and molding the matrix in the form of a biological tissue defect.

23. A method of treating, repairing, or replacing biological tissue comprising administering a composition according to claim 1 to a site of treatment, repair, or replacement in a patient.

24. The method of claim 23 wherein the biological tissue is selected from the group consisting of cartilage, bone, bladder, liver, kidney, and mixtures thereof.

25. A method of providing mechanical integrity to a biological defect comprising implanting a composition according to claim 1 to the site of a biological defect and allowing tissue ingrowth within biodegradable pores of the implanted composition, wherein the implanted composition provides mechanical integrity before, during, and after tissue ingrowth.
